(19) United States
(12) Patent Application Publication
(10) Pub. No.: US 2019/0075762 A1
(43) Pub. Date: Mar. 14, 2019

(54) BREEDING COMB FOR SOLITAIRE BEES

(71) Applicants: DAMIAN KAPKA, Glowno (PL); Przemyslaw KAPKA, Glowno (PL)

(72) Inventors: DAMIAN KAPKA, Glowno (PL); Przemyslaw KAPKA, Glowno (PL)

(21) Appl. No.: 16/083,875

(22) PCT Filed: Mar. 7, 2017
(86) PCT No.: PCT/PL2017/000020

§ 371 Date: Sep. 10, 2018

(30) Foreign Application Priority Data
Mar. 10, 2016 (PK) W.124947

(51) Int. Cl. A01K 47/02; A01K 67/033

(52) U.S. Cl. CPC A01K 47/02; A01K 47/04; A01K 67/033

(57) ABSTRACT

A breeding comb for solitary bees, comprised of single modules in the form of a sheet containing grooves and locks characterised in that, the grooves of the sheet are separated from one another with side walls, while in the lower part the base of the sheet contains lugs that make blocking locks and rabbets in which the side walls of the particular module are inserted, thus forming nesting tunnels with a cross-section with a shape close to a polyhedron, while in the front part the base of the sheet contains a lug and in the rear part the sheet is limited with a rear wall containing a clasp, wherein the external side walls of the sheet are fitted with clasps of the sheet and of the sheet that make side locks, right and left respectively.

This utility model relates to a breeding comb for solitary bees for application in the field of beekeeping.

In the general opinion, bees are above all producers of honey and wax as well as the other bee products. Nevertheless, the most essential role of bees for man is pollination of wild and cultivated plants.

The progressive specialisation in fruit growing, the decreasing number of apiaries with honeybees, as well as the chemicalisation of agriculture all cause the collapse of honeybee colonies make that the problem of crop pollination is becoming more and more essential to stabilization of yields.

It is estimated that bees from breeding colonies cover approximately half of the needs concerning pollination of plants. The remaining gap can be potentially filled by wild pollinating insects—primarily bumblebees and solitary bees, but their population is unfortunately constantly decreasing. Apidae insects are characterised by great species diversity. Solitary bees include the following genera and species: Yellow-faced Bee (Hylaeus), Small Carpenter Bee (Ceratina), Heriades, Small Scissor Bee (Chelostoma), Mason bees (Hoplitis, Osmia), Banded Mud Bee (Chalicodoma), Wood-carving Leaf-cutter (Megachile), Wool Carder Bee (Anthidium). In natural conditions, bees use as nests places like hollow plant stems, e.g. reeds, as well as other kinds of cavities. The great adaptability of some species is evidenced by the fact that they can nest in very unusual places, e.g. hollow bricks or thatched roofs. Nests are of linear character, they consist of successively arranged larval cells. Some species create colonies of nests developing under conditions of easy access to a large number of channels. The gathering of pollen into the nests begins from the end of the channels, which are blanked off, and progresses towards the entrance. Each single cell is filled with pollen by the females and then it is equipped with an egg and separated from the next cell with a species-specific barrier. Depending on the length of the channel, typically several to more than a dozen larval cells are formed in the nest. After sealing the last cell, the entrance to the nest is secured with an appropriate plug that protects the larvae against natural enemies and unfavourable weather conditions.

Solitary bees search for food within a small distance from the nests (approx. 700 m) and they feed on a wide range of foods. Especially remarkable is the complete lack of aggressiveness towards people, which makes it possible to use solitary bees in kitchen gardens.

The traditional rearing of solitary bees makes use of common reed for nests. Disadvantages of the commonly used nesting materials include the greatly labour-consuming preparation of reed nests (i.e. the annually repeated harvesting, selection, defoliation and cutting of reed stems), the risk of allergic sensitisation (when the reeds are torn open and the cocoons are taken out, tiny particles of unused pollen and spores of pathogenic fungi are released to the air), the unfeasibility of removing the remains of cocoons from the nest and carrying out a selection thereof as well as the difficulties with getting completely rid of pests and parasites.

From the descriptions 08020829 and PCT/IB2007/001989, there are known designs relating to a method and an apparatus for breeding and an incubator for solitary bees, which rely on the use of corrugated sheets which folded in half create nesting tunnels. The material used is very susceptible to weather conditions, which has an adverse effect on its durability.

From the utility model description PL67276, there is known a design in which thermoformable foil is used for the production of habitat sheets. Unfortunately, there is a barrier in this method related to the thickness of the walls of the sheets. They are thinner and thus have shorter lifetime. Furthermore, the sheets made according to the described utility model have different thickness at different places and are easily damaged mechanically when the cocoons are removed. Moreover, these sheets can become deformed when exposed to higher temperatures, e.g. under exposure to solar radiation. Additionally, the sheets are overlaid directly one onto another and there is no possibility to connect them permanently.

The objective of the design according to the present utility model is provision of such a nesting habitat for solitary bees that will enable acquisition, without disturbing the natural developmental processes, of bee cocoons or larvae with the same parameters as are acquired in the natural process as well as a reduction in workload and costs related to breeding these bees, without an adverse effect on the insects themselves. Due to the high effectiveness of pollination of cultivated plants, it is of high significance to implement easy and effective ways of breeding, in materials that make alternatives forms of natural nests.

Breeding comb for solitary bees, comprised of single modules in the form of a sheet containing grooves and locks is characterised in that the grooves of the sheet are separated from one another with side walls, while in the lower part the base of the sheet contains lugs that make blocking locks and rabbets in which the side walls of the particular module are inserted, thus forming nesting tunnels with a cross-section with a shape close to a polyhedron while in the front part the base of the sheet contains a lug and in the rear part the sheet is limited with a rear wall containing a clasp that allows for additional stabilisation of the modules connected one on top of another.

The external side walls can be fitted with clasps that make side locks, right and left respectively, that enable serial connection of the selected sheets with one another.

Such construction of sheets containing grooves as well as locks facilitating overlaying of the sheets and also preventing their movement in the horizontal plane, allows joining singular modules (both in the vertical and the horizontal plane) into a form of a wall in which such arranged sheets form one uniform and connected into a whole assembly for breeding solitary bees, filled with nesting tunnels with a cross-section close to a polyhedron. Furthermore, the fact that the side walls are moved away from the beginning of the module makes it possible to create a protrusion (so-called ‘balcony’) on which a bee can effortlessly land and also easily assume the right position to enter the nest (facing forwards or rearwards) depending on the current activity—cleaning the nest, gathering the pollen, laying eggs, building a barrier. An additional advantage of the ‘balcony’ is that it reinforces the structure and makes it more rigid, and also diffuses light, which in turn reduces the temperature inside the nest.

The single breeding combs can be made with the method of injection based on moulds of appropriate shape. Such a way of production provides almost unlimited options as to the colour of the casts. It is possible to add appropriate components that improve resistance to inter alia UV radiation, low or high temperatures, etc. Furthermore, different kinds of plastics can be used in this technology.

It is most advantageous if the breeding comb is made of a plastic where the contact itself between the bees and the artificial material does not adversely affect the process of colonisation of the tunnels, development of the larvae and transformation of the bees into the successive developmental stages.

The module of a package made up of single modules can be enclosed with walls of a wooden nesting box, which additionally protects and isolates the process of colonisation of the nesting tunnels from the external environment and gives the artificial habitats an aesthetic appearance. The utility model has been presented in drawings where

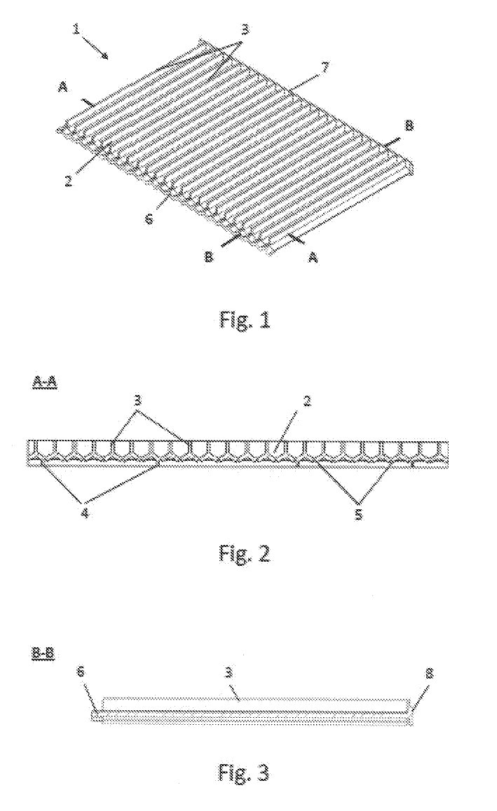

FIG. 1 shows a perspective view of a single module of the breeding comb, with marked cross-sections A-A and B-B;

FIG. 2 shows cross-section A-A of a single module of the breeding comb;

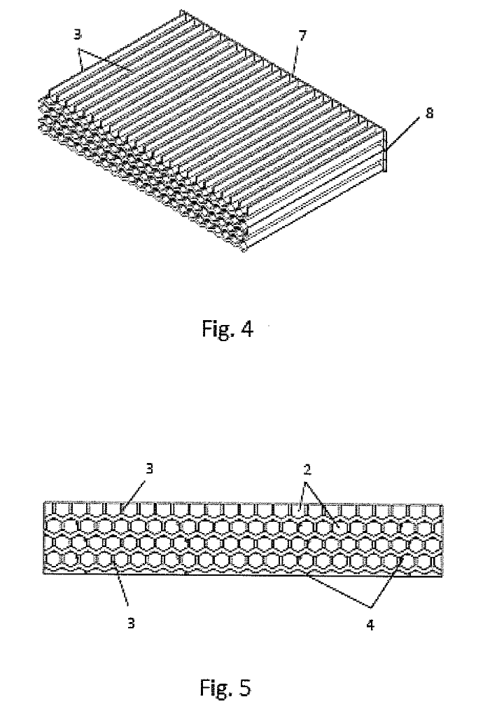

FIG. 3 shows cross-section B-B of a single module of the breeding comb;

FIG. 4 shows a perspective view of four single modules of the breeding comb overlaid one onto another;

FIG. 5 shows a cross-section of four single modules of the breeding comb overlaid one onto another;

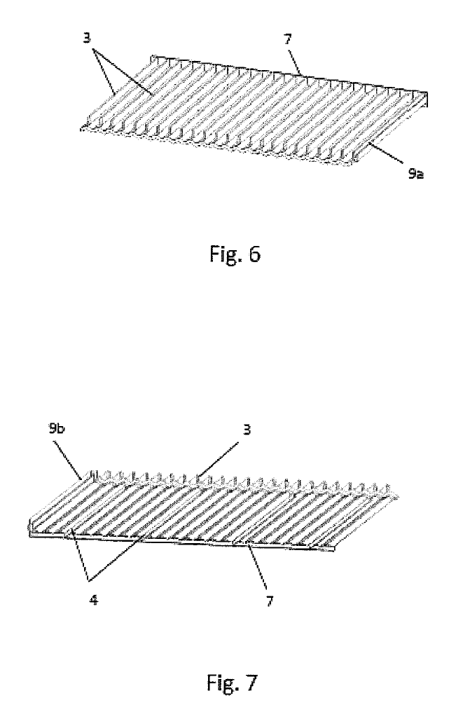

FIG. 6 shows a perspective view of a single module of the breeding comb with a right side lock;

FIG. 7 shows a perspective view of a single module of the breeding comb with a left side lock;

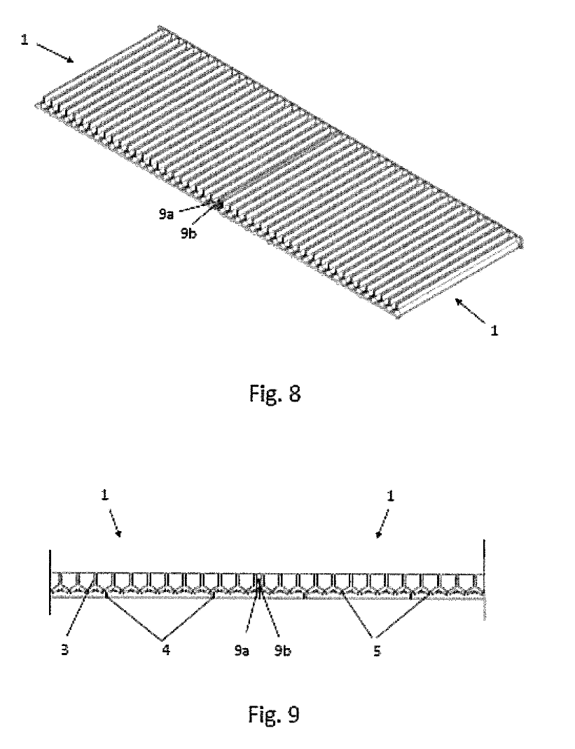

FIG. 8 shows a perspective view of two single modules of the breeding comb connected serially;

FIG. 9 shows a cross-section of two single modules of the breeding comb connected serially.

In a non-limiting example of execution, the breeding comb consists of single modules, each in the form of a sheet 1 comprising grooves 2 separated with side walls 3. In the bottom part, the base of the sheet 1 comprises lugs 4 making blocking locks and rabbets 5 in which the side walls 3 of the bottom module are inserted, thus creating nesting tunnels with a cross-section close to a polyhedron. In the front part, the base of the sheet 1 comprises a protrusion 6. In the rear part, the sheet 1 is limited by a rear wall 7 comprising a clasp 8 enabling additional stabilisation of the modules that are connected one on top of another.

The external side walls 3 of the sheet 1 can be fitted with clasps that make side locks 9a and 9b that make side locks, right and left respectively which make it possible to attach serially another module.

1. A breeding comb for solitary bees, comprised of single modules in the form of a sheet containing grooves and locks characterised in that, the grooves (2) of the sheet (1) are separated from one another with side walls (3), while in the lower part the base of the sheet (1) contains lugs (4) that make blocking locks and rabbets (5) in which the side walls (3) of the particular module are inserted, thus forming nesting tunnels with a cross-section with a shape close to a polyhedron, while in the front part the base of the sheet (1) contains a lug (6) and in the rear part the sheet (1) is limited with a rear wall (7) containing a clasp (8).

2. The breeding comb according to claim 1 characterized in that, the external side walls (3) of the sheet (1) are fitted with clasps of the sheet (9a) and of the sheet (9b) that make side locks, right and left respectively.
